(19) United States
(12) Patent Application Publication
(10) Pub. No.: US 2008/0096255 A1
(43) Pub. Date: Apr. 24, 2008

(54) Method for Preparing Sequence Tags

(76) Inventors: Matthias Harbers, Kanagawa (JP); Yuko Shibata, Kanagawa (JP)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/571,562

(22) PCT Filed: Jul. 2, 2004
(86) PCT No.: PCT/JP04/09862

§ 371 Date: Apr. 24, 2007

(51) Int. Cl. C12P 19/34

(52) U.S. Cl. 435/91.2

(57) ABSTRACT

Means to circulate any nucleic acid molecule and to obtain from such circular nucleic acid molecules fragments that mark both ends of the initial nucleic acid molecule are provided. Means of high value to studies including, but not limited to, expression profiling, splicing, promoter identification, identification of genetic elements, and beyond, which are essential components of commercial applications and services including, but not limited to, drug development, diagnostics, or forensic studies are also provided.

### FIELD OF THE INVENTION

The invention relates to the identification of nucleic acid molecules and cloning of fragments thereof. Information on such fragments can be related to functional regions within genomes or transcribed regions. Furthermore, the invention relates to the analysis of fragments for the purpose of gene identification and expression profiling. Thus, the present invention allows for studies on biological systems, the characterization of genetic elements, and the analysis of genes expressed therein.

### BACKGROUND ART

Genomes contain the essential genetic information for development and homeostasis of any living organisms. For an understanding of biological phenomena, knowledge is required on how such genetic information is utilized in a cell or tissue at a given time point. It is known that mistakes in the utilization of genetic information and related regulatory pathways may cause disease in human or plant and animal in many cases. Thus, a method is needed for expression profiling and annotation of the identified transcripts as well as for characterizing genetic elements under the control of the genetic information. Most expression studies nowadays use either approaches based on in situ hybridization, e.g. microarrays, or those based on high-throughput sequencing of short tags, e.g. SAGE, CAGE, MMPS. The two types of approaches have distinct advantages over each other. However, for our understanding of the regulatory principles behind gene expression, it is desirable to also obtain information on the genetic elements which control gene expression

High-tlhroughput expression profiling is commonly performed by the use of so-called DNA microarrays (Jordan B., DNA Microarrays: Gene Expression Applications, Springer-Verlag, Berlin Heidelberg New York, 2001: Schena A, DNA Microarrays, A Practical Approach, Oxford University Press, Oxford 1999, both hereby incorporated herein by reference). For such experiments specific probes representing individual genes or transcripts are placed on a support and simultaneously hybridized with a plurality of samples. Positive signals are obtained where a probe on the support reacts with a molecule presented with the sample. These experiments allow the parallel analysis of a large number of genes or transcripts. However, the approach is limited to the fact that only genes or transcripts can be studied, which were initially identified by other experimental means. Such means can include cDNA libraries, partial sequence tags and/or results obtained from computer predictions. In the future, the concept of tiled arrays may also allow for an unbiased expression profiling in organisms for which genomic sequences are available (Kapranov P. et al., Science 296, 916-919 (2002), hereby incorporated herein by reference). However, as tiled arrays present genomic sequences as such, data from those experiments are difficult to interpret where multiple transcripts are derived from the same region within the genome. Thus tiled arrays can provide information on which regions within genomes are actively transcripted, but in high-throughput expression profiling experiments fall short on the characterization of individual transcripts.

Due to the limitations of DNA microarray experiments alternative approaches are in use for gene discovery and expression profiling, which are based on partial sequences, said tags, obtained from a plurality of MnRNA samples. The so-called SAGE (Serial Analysis of Gene Expression) method is known as an efficient method for obtaining partial information on the base sequences in mRNAs (Velculescu V. E. et at., Science 270, 484-487 (1995), hereby incorporated herein by reference). This method forms DNA concatemers by ligating multiple short DNA fragments (initially about 10 bp) containing information on the base sequences at the 3′-end of multiple mRNAs, and determines the base sequences in these DNA concatemers. Recently an approved version of SAGE, the so-called LongSAGE, has been published, which allows for the cloning of longer SAGE tags (Saha S. et al., Nat. Biotechnol. 20, 508-12 (2002), US patent applications 20030008290, 20030049653, all hereby incorporated herein by reference). The SAGE method is currently in wide use as an important method for analyzing genes expressed in specific cells, tissues or organisms; and SAGE tags are available for reference in the public domain, e.g. under http://cgap.nci.nih.gov/SAGE.

U.S. Pat. Nos. 6,352,828, 6,306,597, 6,280,935, 6,265,163, and 5,695,934, all hereby incorporated herein by reference, disclosed a different approach for the high-throughput sequencing of short sequence tags, also denoted as Massively Parallel Signature Sequencing or “MPSS”. As described in further details in Brenner S., et al., Nat. Biotechnol. 18, 630-634 (2000), and Brenner S., et al., Proc. Natl. Acad. Sci. USA 97, 1655-1670 (2000), both hereby incorporated herein by reference, preferentially short sequences from the 3′-end of transcripts are obtained in a highly parallel manner performing cycles with different enzymatic reactions on a single layer of beads.

As both of the aforementioned approaches focused on the utilization of 3′-end derived sequence tags, new approaches have been developed to obtain also sequence tags from other regions, in particular the 5′-ends, of transcripts. Such an approach has been disclosed in PCT/JP03/07514, and Shiraki T. et al., Prog. Natl. Acad. Sci. USA 100, 15776-15781 (2003), both hereby incorporated herein by reference. This so-called CAGE (Cap-Analysis-Gene-Expression) approach allows for the cloning of 5′-end specific tags into concatemers similar to the SAGE technology, where the so-called CAGE tags enable not only the detection of transcripts and their expression profiling, but further provide information on transcriptional start sites to allow for mechanistic studies on the regulation of transcription or a higher annotation of transcripts.

However, any of the above approaches focuses only on the cloning and sequencing of one sequence tag per nucleic acid molecule. Such approaches, however, do not always allow for a correct analysis of the information, where often the sequence information within a tag is not sufficient for mapping to the genome or other approaches in bioinformatics. Therefore, it is desirable to not only have a tag from one region within a nucleic acid molecule, but to be able to clone both ends of the nucleic acid molecule in such a way that the tags derived from such an approach would allow for the identification of the ends of nucleic acid molecules.

### SUMMARY OF THE INVENTION

Here, the present invention provides means to circularize any nucleic acid molecule and obtain from such circular nucleic acid molecules fragments that mark the two ends of the initial nucleic acid molecule. Thus, the invention represents a great improvement in the analysis of genomic or transcripted genetic information, and nucleic acid molecules derived thereof The invention provides a further means of high value to studies including, but not limited to, expression profiling, splicing, promoter identification, identification of genetic elements, and beyond, which are essential components of commercial applications and services including, but not limited to, drug development, diagnostics, or forensic studies.

The invention relates to methods for the isolation of fragments from nucleic acid molecules for the purpose of cloning and analysis. Thus, the invention relates to the conversion of a sample containing one or more nucleic acid molecules, and such nucleic acid molecules or any mixture of nucleic acid molecules would be converted into DNA.

In one embodiment the invention relates to the manipulation of nucleic acid molecules that would provides linear nucleic acid molecules containing information on the opposite end sequences of a target nucleic acid molecule in the form of linear double-stranded DNA.

The present invention provides a method for preparing DNA fragments comprising sequences corresponding to two opposite end regions of a linear nucleic acid molecule, comprising the steps of- creating a linear DNA molecule from a nucleic acid molecule; ligating linkers to two opposite ends of the linear DNA molecule, wherein such linkers contain a cloning site and a recognition site for a restriction endonuclease that cleaves at a site outside its recognition site and within the linear DNA molecule; circularizing the linear DNA molecule by closing the linear DNA molecule at its cloning site so as to form a circular DNA molecule; digesting the circular DNA molecule with a restriction endonuclease that cleaves at a site outside its recognition site and cuts out a DNA fragment from the circular DNA molecule, wherein the DNA fragment comprises opposite end regions of the linear DNA molecule; and isolating the DNA fragment.

The invention involves the manipulation of double-stranded DNA by the addition of specific linkers to opposite ends of such a double-stranded DNA molecule, where such linkers would provide a means for the fuirther amplification, manipulation and/or purification of the double-stranded DNA molecule. The linkers as attached to the ends of a double-stranded DNA molecule would provide the necessary means to allow for the circularization of the DNA molecule. Thus, the invention provides a means for the conversion of linear DNA into circular DNA and the amplification of such circular DNA.

Further, the invention involves steps to manipulate DNA fragments in such a way that linkers are attached ends. Such linkers would contain a recognition site for a Class Ils or Class mI enzyme adjacent or close to their cloning sites. Thus, the linkers provide the necessary means to cleave out fragments or tags from the ends of DNA molecules. The invention utilizes the isolation of tags from ends of nucleic acid molecules. Such regions can be derived from different experimental approaches and allow for the characterization of the origin of the initial nucleic acid molecules. Due to the circularization steps, the tags derived from the ends of the same linear DNA molecule are linked to each other by a spacer as derived from linker sequences. Thus, the invention provides a means for the preparation of a new type of sequence tag, the so-called GSC-tag (Gene-Scanning-CAGE-tag), which allows for the identification and characterization of nucleic acid molecules by their end sequences. Furthermore, GSC-tags are prepared in such a way that related tags from the same nucleic acid molecule are combined in the same GSC-tag, and that the spacer sequence connecting the two tags from the ends would allow for the labeling of the GSC-tag by a short sequence tag.

Further, the invention involves the cloning of the tags derived from the DNA molecules. Such tags are purified and cloned as concatemers into tag libraries for easier manipulation and sequencing, said GSC-library. Thus, the invention provides a means for the high-throughput sequencing of tags derived from the ends of nucleic acid molecules.

In an embodiment the invention relates to the cloning of tags from different samples. A label would mark the origin of each molecule within such a mixed tag library. Similarly, tags prepared by different approaches can be individually labeled and used for the preparation of pooled libraries. Thus, the invention relates to the labeling of tags by defined sequences, where such sequences is introduced during the linker ligation and/or circularization steps before cloning into concatemers.

In another embodiment, the invention relates to the sequencing of the tags to allow for their annotation by computational means and their statistical analysis. Thus, the invention relates to a means for gene discovery, gene identification, gene expression profiling, and annotation.

In just another embodiment, the invention relates to the sequencing of the tags to allow for their annotation by computational means and their statistical analysis. Such tags could be derived from regions within genomes. Thus, the invention relates to the characterization of genetic elements within genomes.

In just a different embodiment, the invention relates to the preparation of hybridization probes from the ends nucleic acid molecules. Such regions can be analyzed by the means of in situ hybridization. In a preferred embodiment, the in sits, hybridization experiment makes use of a tiled array.

In just one more embodiment, the invention relates to the full-length cloning of nucleic acid molecules. The sequence information obtained from the tags is used for primer design, and such primers are used to amplify the nucleic acid molecule in an amplification reaction. It is within the scope of the invention to amplify and clone in such a way transcripted regions as well as genomic fragments, where such fragments can contain genetic elements or said promoter regions.

Thus, the invention provides means for the analysis of nucleic acid molecules and short fragments thereof as needed for example for the characterization of biological samples.

### BRIEF DESCRIPTION OF THE DRAWINGS

FIG. 1 is a schematic diargaram showing the first-strand cDNA priming and poly-A tail demoval.

FIG. 2 is a schematic diagram showing the linker ligation step.

FIG. 3 is a schematic diagram showing the amplification step.

FIG. 4 is a schmetic diagram showing the digestion and concatenation steps.

FIG. 5 is a schematic diagram showing the cloning steps

FIG. 6 shows vector pGSC.

FIG. 7 is a diagram showing the targeting of non-polyadenylated RNA.

FIG. 8 is a diagram showing the preparation of hybridization probes.

FIG. 9 shows iii situ hybridization using tiled arrays.

### DETAILED DESCRIPTION OF THE INVENTION

The invention encompasses a method for handling single-stranded as well as double-stranded nucleic acids in the form of linear and circular nucleic acid molecules. Double-stranded DNA means any nucleic acid molecules each of which is composed of two polymers formed by deoxyribonucleotides and in which the two polymers have substantially complementary sequences to each other allowing for their association to form a dimeric molecule. The two polymers are bound to one another by specific hydrogen bonds formed between matching base pairs within the deoxyribonucleotides. Any DNA molecule composed only of one polymer chain formed by two or more deoxyribonucleotides having no matching complementary DNA molecule to associate with is considered to be a single-stranded DNA molecule for the purpose of the invention, even if such a molecule may form secondary structures comprising double-stranded DNA portions. As used interchangeably herein, the terms “nucleic acid molecule(s)” and “polynucleotide(s)” include RNA or DNA regardless of single or double-stranded, coding or non-coding, complementary or not, and sense or antisense, and also include hybrid sequences thereof. In particular, it encompasses genomic DNA and complementary DNA which are transcribed or non-transcribed, spliced or not spliced, incompletely spliced or processed, independent from its origin, cloned from a biological material, or obtained by means of synthesis. RNA for the purpose of the invention is considered a single-stranded nucleic acid molecule even where such a molecule may form secondary structures comprising double-stranded RNA portions. In particular, RNA encompasses for the purpose of the invention any form of nucleic acid molecule comprised of ribonucleotides, and does not related to a particular sequence or origin of the RNA Thus, RNA can be transcribed in vivo or in vitro by artificial systems, or non-transcribed, spliced or not spliced, incompletely spliced or processed, independent from its natural origin or derived from artificially designed templates, mRNA, tRNA, rRNA, obtained by means of synthesis, or any. mixture thereof. More precisely, the expressions “DNA”, “RNA”, “nucleic acid”, and “sequence” encompass nucleic acid materials themselves and are Thus, not restricted to particular sequence information, vector, phagemid or any other specific nucleic acid molecule. The term “nucleic acid” is also used herein to encompass naturally occurring nucleic acids, artificially synthesized or prepared nucleic acids, any modified nucleic acids into which at least one or more modifications have been introduced by naturally occurring events or through approaches known to a person skilled in the art. Similarly, a “tag” according to the invention can be any region of a nucleic acid molecules as prepared by the means of the invention, where the term “tag” as used herein encompasses any nucleic acids fragment, no mater whether it is derived from naturally occurring, artificially synthesized or prepared nucleic acids, any modified nucleic acids into which at least one or more modifications have been introduced by naturally occurring events or through approaches known to a person skilled in the art. Furthermore, the term “tag” does not relate to any particular sequence information or their composition but to the nucleic acid molecules as such. The terms “purity”, “enriched”, “purification”, “enrichment”, or “selection” are used interchangeably herein and do not require absolute purity or enrichment of a product but rather are intended as relative definitions. The terms “specific”, “preferable”, or “preferential” are used interchangeably herein and do not require absolute specificity of a DNA or RNA hybridization probe, or an enzyme for its substrate or an activity, but rather they are intended to have relative definitions which include the possibility that an enzyme may have low or lower affinity to other compounds related or unrelated to its substrate. Similarly, the terms used to name an enzyme, or an enzymatic activity, are used herein to describe the finction or activity of such a component, but do not require the absolute purity of such a components. Thus, any mixture containing such an enzyme, enzymatic activity, or mixtures thereof with other components of the same, related or unrelated function are within the scope of the invention. Similarly, DNA or RNA molecules may function in a specific manner as hybridization probes, and as such are related to as “complementary sequences” for the purpose of the invention, or in experiments where such probes are applied for the detection of a related nucleic acid molecule, even where such a probe and the target molecule may be distinct by naturally occurring or artificially introduced mutations in individual positions. The term “biological samples” includes any kind of material obtained from living organisms including microorganisms, animals, and plants, as well as any kind of infectious particles including viruses and prions, which depend on a host organism for their replication. As such “biological samples” include any kind material obtained from a patient, animal, plant or infectious particle for the purpose of research, development, diagnostics or therapy. Thus, the invention is not limited to the use of any particular nucleic acid molecules or their origin, but the invention provides general means to be applied to and used for the work on and the manipulation of any given nucleic acid. Any such nucleic acid molecules as applied to perform the invention can be obtained or prepared by any method known to a person skilled in the art including, but not limited to, those described by Sambrook J. and Russuell D. W., Molecular Cloning, A Laboratory Manual, Cold Spring Harbor Laboratory Press, New York, 2001, hereby incorporated herein by reference.

The invention relates to methods for the isolation of fragments from nucleic acid molecules for the purpose of cloning and analysis. Thus the invention relates to the conversion of a sample containing one or more nucleic acid molecules, where such nucleic acid molecules or any mixture of nucleic acid molecules would be converted into DNA To perform the invention, nucleic acid molecules can be derived from any naturally occurring genomic DNA, RNA sample, an existing DNA library, is of artificial origin, or any mixture thereof. The invention is not limited to the use of an individual nucleic acid molecule or any plurality of nucleic acid molecules, but the invention can be performed on an individual nucleic acid molecule or any plurality of nucleic acid molecules regardless whether such pluralities would occur in nature, be derived from an exciting library, or be artificially created. Furthermore, the invention can process any nucleic acid molecule regardless of its origin or nature. Thus it is within the scope of the invention that the nucleic acid molecules could be full-length molecules as compared to naturally occurring nucleic acid molecules, or any fragment thereof Even furthermore, it can be envisioned that such fragments of nucleic acid molecules could be prepared by a random process or by a targeted dissection of nucleic acid molecules by the means of an enzymatic activity with a preference for a certain sequence, or by means which would allow for the fragmentation based on the structure of the nucleic acid molecule including, but not limited to, exons and introns within transcripted regions. Thus the invention is not restricted to the use of any particular starting material.

The invention is not dependent on the use of DNA only, as a person familiar with the state of the art will know different approaches to convert RNA into DNA including, but not limited to, those approaches disclosed by Sambrook J. and Russuell D. W., ibid, hereby incorporated herein by reference. After conversion of RNA into DNA, a single-stranded or double-stranded DNA molecule having the same or complementary sequence to the original RNA can be obtained, said cDNA. Such cDNA molecules are commonly prepared in the form of liner DNA, where the two open ends allow for their manipulation. However, even where cDNAs are cloned into a vector, a person trained to the state of the art will know about the necessary means to release an insert from such a vector to convert it into linear DNA.

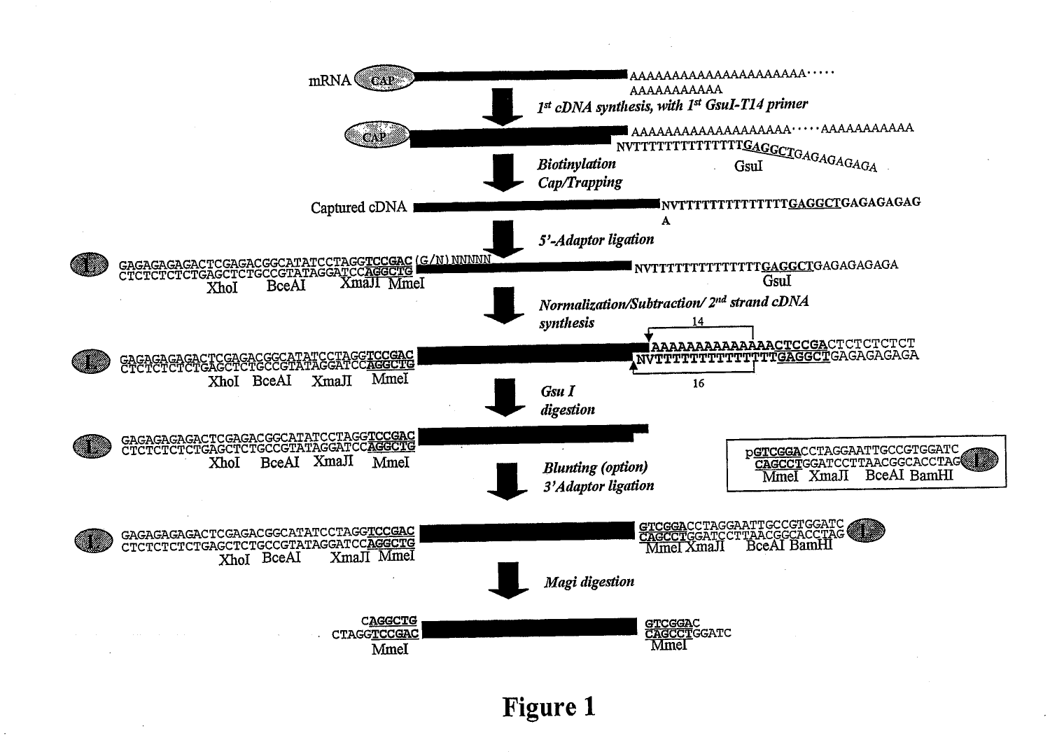

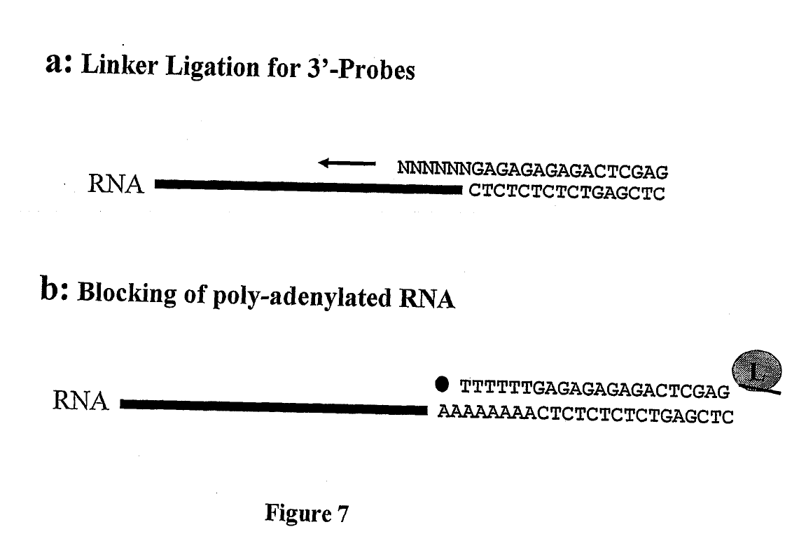

In one embodiment of the invention, parts of the sequencing tags are derived from the 3′-end of transcripts. For the cloning of tags derived from the actual 3′-end of mRNAs, it is important to remove polyA-tails from the RNA to obtain meaningful information. One approach for the removal of polyA tails has been published by Shibata Y. et al., Biotechniques, 1042 to 1044, 1048-1049 (2001), hereby incorporated herein by reference, which can be applied for the cloning of 3′-end related tags (compare to FIG. 1). The primer as used for the first-strand cDNA synthesis has a recognition site for the Class IIs restriction enzyme GsuI, which will cleave the resulting double-stranded cDNA 14/16 bp from its recognition site, which is adjacent to an oligo-dT stretch of 14 nucleotides used in the priming step. After cDNA synthesis GsuI is used to cut of the remaining poly-dA/dT stretch between the 3′-end of the cDNA and its recognition site. The cohesive end created by Gsu I digestion can then be used for 3′-end-specific linker ligation, where such a linker could contain a Class IIs or Class III recognition site adjacent or close to the ligation site for cutting of a sequencing tag, a cloning site, and/or a label for the purification of such a tag. Thus the invention provides means for the removal of polyA-tails from 3′-ends to allow for a meaningful analysis of mRNAs. In just a different embodiment, the invention provides means for the 3′-end specific priming of non-polyadenylated RNA In this embodiment of the invention, a double-stranded linker having a random single-stranded overhang is ligated to the 3′-end of a RNA molecule (FIG. 7a). Such linkers can be designed similar to other approaches known to a person familiar with the state of the art including but not limited the method described by Shibata Y. et al., Biotechnique 30, 1250-1254 (2001), hereby incorporated herein by reference. The 3′-end specific linker as used for the priming of the cDNA synthesis, could further contain a Class IIs or Class III recognition site for cutting of the sequencing tag from the 3′-end of the ligation product, a cloning site, and/or a label for the purification of such a tag. Thus the invention provides means for the—possibly—full-length cDNA preparation from non-polyadenylated RNA. Furthermore, the same linker ligation step can be applied to block the cDNA synthesis of polyadenylated RNA. In such an embodiment of the invention, a double-stranded linker having a single-stranded oligo-dT overhang is ligated to the 3′-end of a RNA molecule (FIG. 7b). Due to the oligo-dT overhang, such a linker would preferentially be ligated to polyadenylated RNA. However, in contrast to the aforementioned linker having random overhangs, the 3′-end of the oligo-dT overhang would be blocked, for example by the use of a dideoxy nucleotide in the last position. Thus, such a modified linker would no longer enable strand extension. In addition the 5′-end of the upper strand of such a linker could be modified in such a way that a specific binding substance would be attached to it, where such a specific binding substance would allow for the selective removal of polyadenylated RNA by the means of a high affinity ligand binding to the specific binding substance. Many combinations of a specific binding substance and a high affinity ligand are known to a person familiar with the state of the art including, but not limited to, the use of biotin and streptavidin, or digoxigenin and an anti-digoxigenin antibody. In this way, the invention provides means for the selective priming of non-polyadenylated RNA, and the separation of such RNA from polyadenylated RNA- Thus the invention provides means for the cloning and analysis of real 3′-ends of nucleic acid molecules including any type of RNA.

In a different embodiment of the invention, the sequencing tags are obtained from the 5′-end of transcripts. Different approaches for the utilization of 5′-end-specific sequence tags have been disclosed in PCT/JP03/07514, and Shiraki T. et al., ibid, both hereby incorporated herein by reference. All such approaches make use of the 5′-end-specific cap structure of mRNA molecules, which can be used to selectively enrich 5′-ends or full-length mRNA molecules. As well known to a person familiar with the state of the art of the field, such approaches include but are not limited to the cap trapper method (Carninci P. et al., Methods in Enzymology, 303, pp. 1944, 1999, hereby incorporated herein by reference), oligo-capping (Maruyama K., Sugano S., Gene 138, 171-174 (1994), hereby incorporated herein by reference), use of a cap-binding protein Edery I. et al., Mol Cell Biol. 15, 3363-3371 (1995), hereby incorporated herein by reference), use of an antibody that specifically binds to the cap structure (Theissen H. et al., EMBO J. 12, 3209-3217 (1986), hereby incorporated herein by reference), oxidation of cap structure followed by adding an oligonucleotide to the cap structure (U.S. Pat. No. 6,022,715, hereby incorporated herein by reference), or the cap-switch method disclosed in U.S. Pat. No. 5,962,272, hereby incorporated herein by reference. Any of the aforementioned approaches allows for the selection of the 5′-ends, followed by the ligation of a linker to the 5′-end of transcripts, where such a linker would contain a Class IIs or Class III recognition site for cutting of a sequencing tag, a cloning site, and/or a label for the purification of such a tag. Thus in this embodiment of the invention, the cap-structure would be used to direct the linker, and to assure the capturing of full-length transcripts. Thus the invention provides means for capturing true 5′-ends of transcripted regions.

In one embodiment the invention relates to the manipulation of nucleic acid molecules, where such nucleic acid molecules would be prepared in the form of linear double-stranded DNA. Such double-stranded DNA can be derived from RNA, and be prepared according to any of the aforementioned approaches, or can be taken from any other source, which allows for the isolation of double-stranded or single-stranded DNA from resources including, but not limited to, genomic DNA, cDNA, cloned DNA or any fragment or mixtures thereof. Thus the invention is not limited to a certain source of nucleic acid, but any nucleic acid molecule as such or any mixture of thereof can be applied to perform the invention. Furthermore, as the invention can be applied to the use of single-stranded RNA and DNA, it is within the scope of the invention to manipulate the complexity of single-stranded nucleic acid molecules by the means of subtraction, normalization or selective enrichment by any of the methods known to a person trained to the state of the art including, but not limited to, the approaches published by Carninci P. et al., Genome Res. 10, 1617-1630(2000), hereby incorporated herein by reference (compare FIG. 1). Independent from the starting material used to perform the invention, the single stranded first-strand cDNA material can be fractionated by means of subtractive hybridizations and physical separation to allow for enrichment of nucleic acid molecules of differentially expressed genes or for the concentration of transcripts of low abundance. Thus the invention relates to means on how to process pluralities of nucleic acid molecules for the purpose of their analysis and cloning.

In just a different embodiment, the invention relates to the manipulation of double-stranded DNA by the addition of specific linkers to both ends of such a double-stranded DNA molecule, where such linkers would provide means for the further amplification, manipulation and/or purification of the double-stranded DNA molecule. Such a linker or linkers can be directly attached to double-stranded DNA in a ligation reaction, be introduced by the ligation of a double-stranded linker having a single-stranded overhang to single-stranded DNA, or be introduced as part of the primer used to drive the DNA synthesis from a RNA or DNA template. The linkers as attached to the ends of a double-stranded DNA molecule would be preferable of double-stranded DNA. Any such linker independently of the way of usage or the way it was introduced or attached to the nucleic acid molecule would contain certain features for the manipulation of the double-stranded DNA molecule. Such features could include, but are not be limited, recognition sites for restriction endonucleases, region complementary to primers used in an amplification reaction, and labeling with selective binding substances including, but not limited to, biotin or digoxigenin. Furthermore, such linker can contain information for the labeling of the attached DNA molecules, where such a label would be encoded be a short sequence within one or both linker molecules, and a recognition site for an endonuclease, which cleaves outside of its recognition sites. In a preferable embodiment, such a recognition site would be adjacent to the junction point between the nucleic acid molecule and the linker. In a different embodiment, such a recognition site would be close or very close to the junction point between the nucleic acid molecule and the linker, where the recognition site and the nucleic acid molecule would be separated by one (1), two (2), three (3), four (4), five (5) or even six (6) nucleotides. In a preferable embodiment, the endonuclease, which cleaves outside of its recognition sites, is a Class IIS or a Class III enzyme. In an even more preferable embodiment, the endonuclease, which cleaves outside of its recognition sites, is one out of Gsu I, MmeI, BpmI, BsgI, or EcoP15I. Thus the invention provides means for the labeling of nucleic acid molecules, in particular where nucleic acid molecules of different origin are mixed for the purpose of their analysis or cloning, where such labels are introduced by a linker or are derived thereof.

In just one more embodiment, the linkers as attached to the ends of a double-stranded DNA molecule would provide the necessary means to allow for the circularization of the DNA molecule. Here the invention relates to the isolation of tags from ends of nucleic acid molecules, where such regions can be derived from different experimental approaches and allow for the characterization of the origin of the initial nucleic acid molecules. Due to the circularization steps, the tags as derived from the ends of the same linear DNA molecule are linked to each other by a spacer as derived from linker sequences. Thus the invention provides means for the preparation of a new type of sequence tag, the so-called GSC-tag (Gene-Scanning-CAGE-tag), which would allow for the identification and characterization of nucleic acid molecules by their end sequences. Furthermore, GSC-tags are prepared in such a way that related tags from the same nucleic acid molecule are combined in the same GSC-tag, and that the spacer sequence connecting the two tags from the ends would allow for the labeling of the GSC-tag by a short sequence tag. Therefore the circularization step is an essential part of the invention, as only by connecting the ends of the nucleic acid molecule, it can be assured that both ends from the same molecule would be cloned into the same GSC-tag. Alternatively, it can be envisioned that the circularization of a nucleic acid molecule can be achieved by cloning into a vector, where the resulting vector construct would be comprised of circular DNA. Where such a vector would provide the necessary means for the isolation of tags derived from the ends of the insert, it could be foreseen that after cutting out the central part of the insert, the tags could be directly ligated to each other using the backbone of the vector as a spacer to link tags as derived from the same nucleic acid molecule, said insert. After the ligation of the two tags by self-ligation of the ends of the vector, such GSC-tags as comprised of the tags from both ends of the insert, said nucleic acid molecule, could be cut out of the vector and further processed according to the invention. Thus it is within the scope of the invention to use a vector or an unrelated nucleic acid molecule to perform the circularization step, where such a vector or nucleic acid molecule would function as a spacer. The use of a vector or an unrelated nucleic acid molecule can be advisable, where the linear DNA molecule, said nucleic acid molecule, may not allow for direct circularization, for example due to restrictions by its length. However, for many or most applications it can be preferable to directly circularize the linear DNA molecule, said nucleic acid molecule, using cloning sites as provided by the linkers, since the direct circularization would reduce the number of steps to perform the invention.

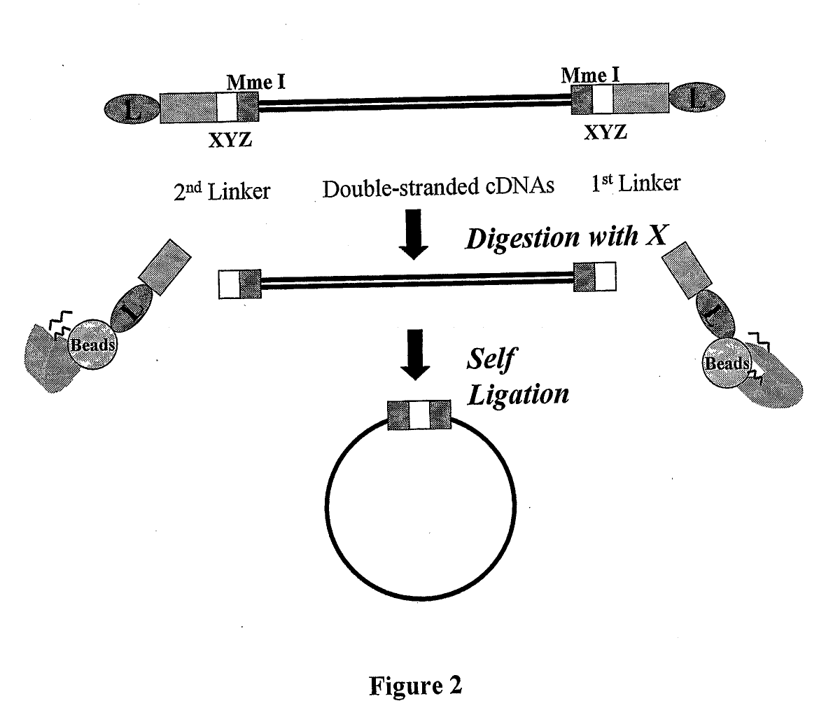

The circulation reaction can make use of blunt ends or cohesive ends depending on the experimental needs. In a preferable embodiment of the invention the linkers at both ends of the nucleic acid molecule have recognition sites for the same restriction endonuclease or isoschizomers creating the same cohesive ends or blunt ends to allow for the recombination of these ends (compare FIG. 2). In such an experiment, parts of the linker sequences would be cleave of to create the cohesive ends for self-ligation. In a different embodiment, the ends of the linkers, as released after the digestion with the restriction endonulcease, would have selective binding substances attached to them, which would allow for their separation from the nucleic acid molecules by the means of a high affinity binding substance. Such pairs of selective binding substances and high affinity binding substances include but are not limited to the combination of biotin-labeling of nucleic acid molecules and binding to avidin or streptavidin, or the use of digoxigenin and an antibody directed against digoxigenin. Both systems provide convenient means for the separation of free nucleic acid molecules and labeled linker fragments, where such fragments can be easily removed by attaching the high affinity binding substance to an insoluble matrix. Many protocols are known to a person trained to the state of the art for the use of an insoluble matrix for the separation of labeled nucleic acid molecules from non-labeled nucleic acid molecules. In a different embodiment of the invention, the nucleic acid molecule has been prepared in such a way that it is resistant to cleavage by the restriction endonulclease used for digesting the linkers. Such a protection can be achieved for example by the incorporation of modified nucleotides during the chemical or enzymatic synthesis of such nucleic molecules, or by the later modification of such nucleic acid molecules by the means of a methyltransferase. Many matching pairs of restriction endonucleases and methyltransfereases are known to a person trained to the state of the art in the field, which could be applied here, including, but not limited to, those commercially available from New England BioLabs (http://www.neb.comn/nebecomin/default.asp, the product documentation as provided at their homepage is hereby incorporated herein by reference) or Fermentas (http://www.fermentas.com/, the product documentation as provided at their homepage is hereby incorporated herein by reference). Furthermore, it is within the scope of the invention to perform the circularization of the nucleic acid molecules by the means of a recombinase, or overlap extension reactions. In a different embodiment, the circularization step could be preformed by the means of a recombinase, where the linkers would provide the necessary means to allow for the recombination step. A person trained to the state of the art is familiar with many recombination systems, which could be applied here. In particular the Cre (Causes REcombination) recombinase from the bacteriophage PI, which catalyzes the recombination between two identical double stranded loxP sites (Locus Of crossover (X) in P1 sites), is widely used as a valuable tool, where it is a great advantage that the Cre/loxP system finctions without any co-factors or additional sequence elements allowing for effective recombination in vito. The Cre recombinase mediated step can be performed on purified DNA where such DNA will be incubated directly with the enzyme. Purified Cre recombinase can be obtained from different suppliers including CLONTECH (BD Biosciences, Palo Alto, Calif., USA), Novagen (Madison, Wis., USA), and New England BioLabs (Beverly, Mass., USA), the maker's instructions and documentations on all of them are hereby incorporated herein by reference. Thus the invention provides means where by the use of different restriction endonucleases or recombinases a linear DNA molecule is converted into circular DNA molecule. The circularization step brings the ends of the linear DNA molecule, said nucleic acid molecule, together to allow for the preparation of GSC-tags holding sequence information on both ends of the linear DNA molecule, said nucleic acid molecule, and having a linker-derived spacer region, where such a spacer could contain elements to label its origin by a sequence tag. The circularization step allows further for the labeling of nucleic acid molecules, and where the recognition sequence of the restriction endonuclease would function as a sequencing tag after the formation of the circular nucleic acid molecule. Thus the invention provides means for the conversion of linear DNA into circular DNA for the purpose manipulation of the ends of a linear DNA molecule.

In another embodiment of the invention, remaining linear DNA is removed from circular DNA after the circularization reaction by the means of an exonuclease. Such an exonuclease should have a much higher activity for linear DNA as compared to circular DNA- One example for such an exonuclease could be exonulcease m (available from Fermentas, #EN0191, http://www.fermentas.com/, the product documentation to it is hereby incorporated herein by reference) or exonulcease I (available from Fermentas, #EN0581, http://www.fermentas.com/, the product documentation to it is hereby incorporated herein by reference), but there are many more exonucleases known to a person familiar with the field, which could be applied for this step. Thus the invention provides means for the removal of nucleic acid molecules, which failed in the self-ligation reaction, and to enrich for circular nucleic acid molecules over linear nucleic acid molecules.

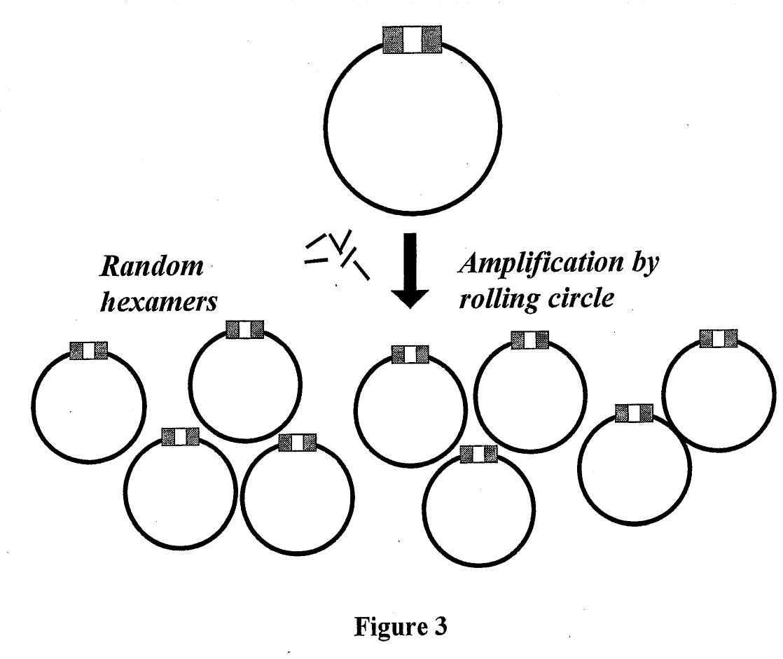

In a different embodiment of the invention the circular DNA is used in an amplification reaction. Many approaches are known to a person trained to the state of the art in the field for the amplification of circular DNA including, but not limited to, the use of the so-called “rolling circle” amplification. As shown in FIG. 3, the amplification of the circular DNA for the purpose of the invention is preferentially done by the means of a rolling circle amplification reaction making use of random primers including, but not limited to, the use of hexamers, and a DNA polymerase with a strong strand-replacement activity including, but not limited to, Phi29 DNA polymerase. Such an amplification reaction for example can be performed by the TempliPhi™ DNA Amplification Kit from Amersham Biosciences (Cat. No. 25-6400-10, the handbook of which is hereby incorporated herein by reference). This kit and any similar isothermal amplification reaction provides very effective means for the amplification of circular DNA over linear DNA, as linear DNA cannot function as a template for rolling circle amplification reactions. Thus the invention provides means for the selective amplification of circular DNA over linear DNA to make circular DNA available for further manipulation.

Further, the invention relates to steps to manipulate DNA fragments in such a way that the linkers attached to the ends of a nucleic acid molecule, and as used in the circularization step, would contain one or more recognition sites for a Class IIs or Class III enzyme adjacent or close to their cloning sites, said the nucleic acid molecule. In a preferable embodiment, the Class IIs enzyme would be GsuI, in a more preferable embodiment, the Class IIs enzyme would be MmeI, and in an even more preferable embodiment, the Class III restriction enzyme would be EcoP15I. Thus the length of the tags as cut off from the ends of the DNA molecule may vary dependent on the restriction enzyme used to create them. Furthermore, it is within the scope of the invention, that different enzymes are used for the digestion at the 3′- and the 5′-end, and that the 3′-end and 5′-end related tags have a different length. Therefore tags as derived from the ends of a DNA molecule, said nucleic acid molecule, may have a length of ten to fifteen (10-15), fifteen to twenty (15-20), twenty to twenty-five (20-25), or twenty-five to thirty (25-30) bp. Just as an example, in the case of using the preferable enzyme MmeI, the tags would be some 16/18 bp in length. Thus the linkers would provide the necessary means to cleave out fragments, said tags, from the ends of such DNA molecules. Thus the invention relates to the isolation of tags from ends of nucleic acid molecules, where such tags could be used for the identification and characterization of the nucleic acid molecule, from which the tags are derived. In a preferable embodiment of the invention such tags are isolated from the nucleic acid molecules after the self-ligation step. In this embodiment, the fragments as released by digestion with the Class IIs or Class III enzyme would be comprised of tags derived from both ends of the nucleic acid molecule linked to each other by sequences derived from the linkers. Thus the invention provides means for the isolation of sequencing tags from both ends of a nucleic acid molecule, where the two tags as derived from the same nucleic acid molecule would be attached to each other via a spacer as derived from the linkers. As the connecting linker sequences comprise the recognition site used in the circularization step, the linker would further contain a sequencing tags for labeling the origin of the tags in pluralities of nucleic acid as obtained from different samples.

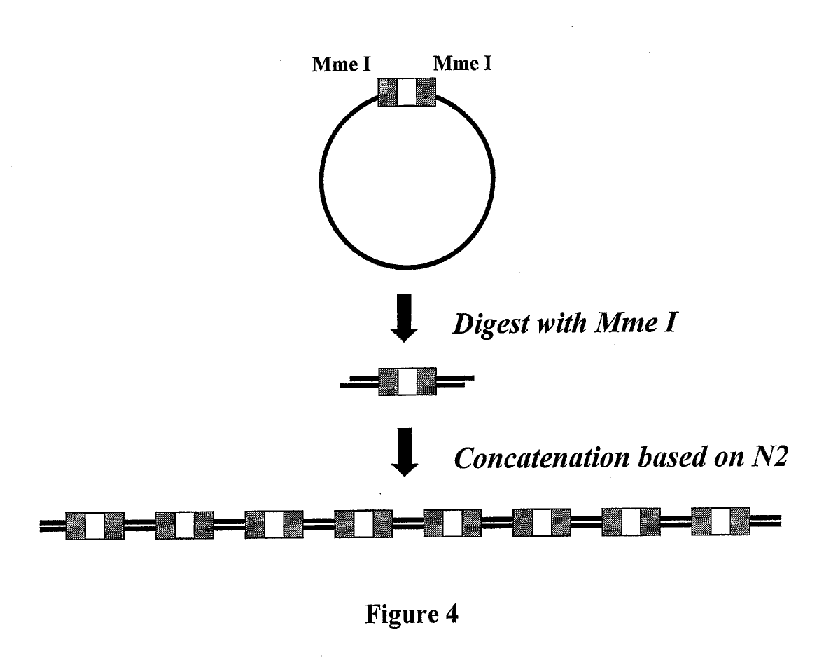

In a different embodiment, the invention relates to the cloning of the tags as derived from both ends of DNA molecules, said GSC-tags, where such tags are purified and cloned into concatemers, and where such concatemers are cloned into libraries for easier manipulation and sequencing (FIG. 4). In a preferable embodiment, the digestion step with the Class IIs or Class III enzyme creates cohesive ends for the ligation of different tags to each other. For example for the use of MmeI, the enzyme would create N2 overhangs, where N2 would allow for 16 different combinations. Therefore for the use of complex samples as comprised of pluralities of nucleic acid molecules, 16 different combinations would allow for the cloning of tags into concatemers. Reaction conditions for concatenation reactions on mixtures of tags prepared by the use of MmeI are known to a person trained to the state of the art in the field including, but not limited to, protocols used for the preparation of Di-Tags within of Long-SAGE libraries (WO 02/10438 A2, hereby incorporated herein by reference). In a different embodiment, the ends created by the digestion with the Class IIs or Class III enzyme are converted into blunt ends, and the concatenation reaction makes use of the ligation of blunt ends. Many different approaches are known to a person trained to the state of the art for the blunting of DNA including, but not limited to, those described by Sambrook J. and Russuell D. W., ibid, hereby incorporated herein by reference. Thus the invention provides means for the assembly of tags into concatemers for the purpose of high-throughput sequencing of tags as derived from the ends of nucleic acid molecules, said GSC-tags.

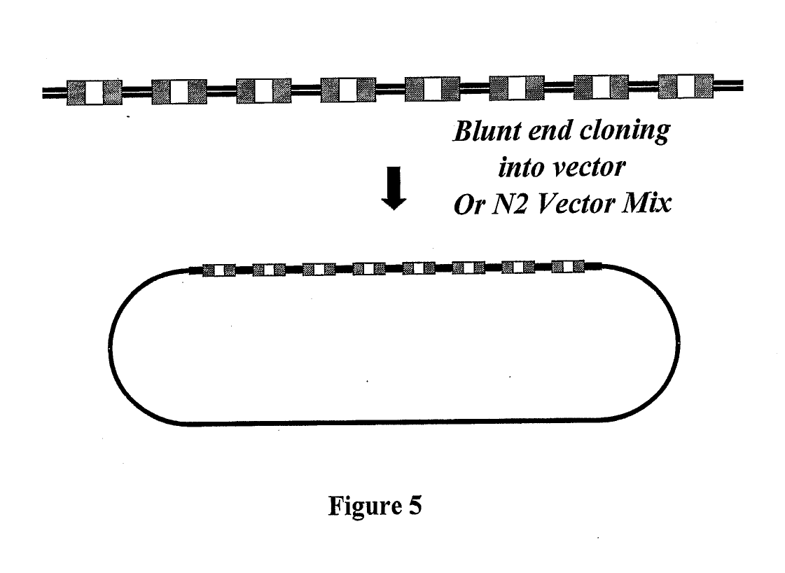

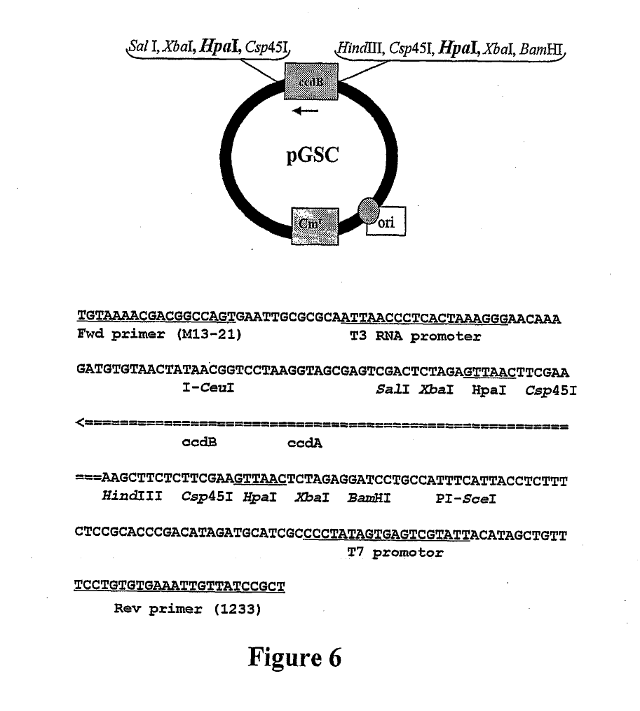

In another embodiment of the invention, the concatemers are cloned into a vector to prepare a library (FIG. 5). For the cloning into the vector, matching recombination sites can be used as used in the concatenation reaction, or the concatemers could be blunted at their ends to allow for cloning into a vector. Many different approaches are known to a person trained to the state of the art for the blunting of DNA and the ligation of blunt ends.including, but not limited to, those described by Sambrook J. and Russuell D. W., ibid, hereby incorporated herein by reference. In a preferable embodiment of the invention the concatemers would be cloned into the vector pGSC (FIG. 6), which provides different cloning sites for the use of cohesive or blunt ends. In a different embodiment of the invention linkers are attached to the ends of the concatemers, where such linkers would provide priming sites for the amplification of the concatemers and/or cloning sites for the cloning of the concatemers into a vector. It is within the scope of the invention, to use such linkers to introduce recombination sites for the cloning of the concatemers by the means of a recombinase rather than using classical means such as restriction endonucleases including, but not limited to, rare cutters and a ligase. In one example, the cloning of the concatemers could be performed by the Gateways System from Invitrogen (http:www.invitrogen.com/, the information to which as provided on their homepage is hereby incorporated herein by reference). In a more preferable example, the Gateway® BP Clonase™ Enzyme Mix from Invitrogen (Cat. No. 11789-013, the product information on which is hereby incorporated herein by reference) is used to clone the PCR products comprising the concatemer into a target vector. In just a different embodiment the invention relates to the cloning of tags from different samples into a library, where a label would mark the origin of each molecule within such a mixed tag library. Similarly, tags as prepared by different approaches can be individually labeled and used for the preparation of pooled libraries, where—as explained above - sequences derived from the linkers would function as a label of each tag. Furthermore, in cases where linkers have been used for the cloning and/or amplification of the concatemers, such terminal linkers could introduce sequence tags to mark concatemers and their origin. Thus the invention relates to the preparation of libraries with the option to the labeling of tags by defined sequences, where such sequences would be introduced during the linker ligation steps before cloning into libraries.

In a different embodiment, the invention provides means for the analysis of concatemers by sequencing in combination with computational analysis. Regions as derived from linkers would in such an application provide information on the origin and the orientation of the sequencing tags within the concatemer, as compared to the regions derived from the ends of the nucleic acid molecule. As the structure of the GSC-tag is known, computational means would allow for the identification of the different regions within the GSC-tag, such as those derived from the nucleic acid molecule and those derived from the linker. The sequencing tags as such would be further analyzed and annotated by the computational methods including, but not limited to, the mapping to genomic sequences, alignments to sequence information within the public domain including those on transcribed regions, alignments against each other, or statistical analysis on GSC-tag frequencies within libraries, including, but not limited to, the applications disclosed in PCT/JP03/15956, PCT/JP03/07514 and WO 02/10438, all hereby incorporated herein by reference. Thus the invention provides different means for the analysis of nucleic acid molecules for example for their expression in a biological sample, or for example for their contribution to a given cDNA library.

In just another embodiment, the invention relates to the sequencing of the tags to allow for their annotation by computational means and their statistical analysis, where such tags would be derived from regions within genomes. It is within the scope of the invention to prepare fragments from genomic DNA, and to characterize such fragments by sequencing tags derived from the ends of such fragments of genomic DNA. In one embodiment such genomic DNA fragments could be obtained from regions bound to DNA binding proteins. One approach for the identification of targets for distinct DNA binding molecules is the so-called “Chromatin Immunoprecipitation” (ChIP), where in vivo DNA binding molecules are cross-linked to their binding sites within genomic DNA by treatment with formaldehyde (Kuras L., Methods Mol. Biol. 284, 147-162 (2004), hereby incorporated herein by reference). After immunoprecipitation of the protein-DNA complexes with specific antibodies targeted against such a DNA binding molecules, DNA fragments can be amplified from such complexes by any method known to a person trained to the state of the art in the field, and forwarded to cloning of tags from both ends of such genomic fragments by the means of the invention. Similar information can further be obtained by the dam methyltransferase assay, which applies fusion proteins of the dam methyltransferase and DNA binding factors. The DNA-binding domain of the DNA binding factor as part of the fusion protein will tether the dam methyltransferase to specific binding sites in the genome, which results in adenine niethylation at the binding site. Isolated genomic DNA can then cleavsed by the methylation-dependent restriction endonuclease DpnI, and DNA fragments are isolated for analysis (van Steensel B. and Henikoff S., Nat. Biotechnol. 18, 424428 (2000), and van Steensel B. et al., Nat. Genet. 27, 304-308 (2001), both hereby incorporated herein by reference). Similar to genetic fragments obtained by ChIP, those fragments can be applied to perform the invention. Thus the invention relates to the characterization of genetic elements within genomes, where such elements could be analyzed by computational means such as mapping to a genome or alike.

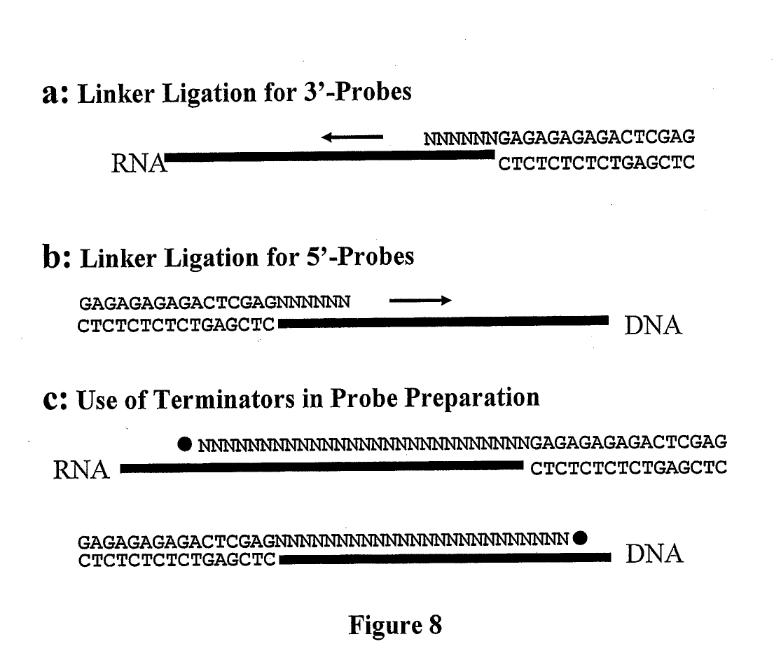

In just a different embodiment, the invention relates to the preparation of hybridization probes from the ends nucleic acid molecules, where such regions would be analyzed by the means of in situ hybridization (FIG. 8). Thus the invention provides means for the confirmation of the boarders of nucleic acid molecules by independent means, where the hybridization probes could be prepared by ligation of linkers to the ends of a nucleic acid molecule, and where such linkers would be used for the preparation of hybridization probes. In a different embodiment of the invention sequences as derived from the tags would be used for primer design, where such primers could be used to drive the preparation of the hybridization probes.

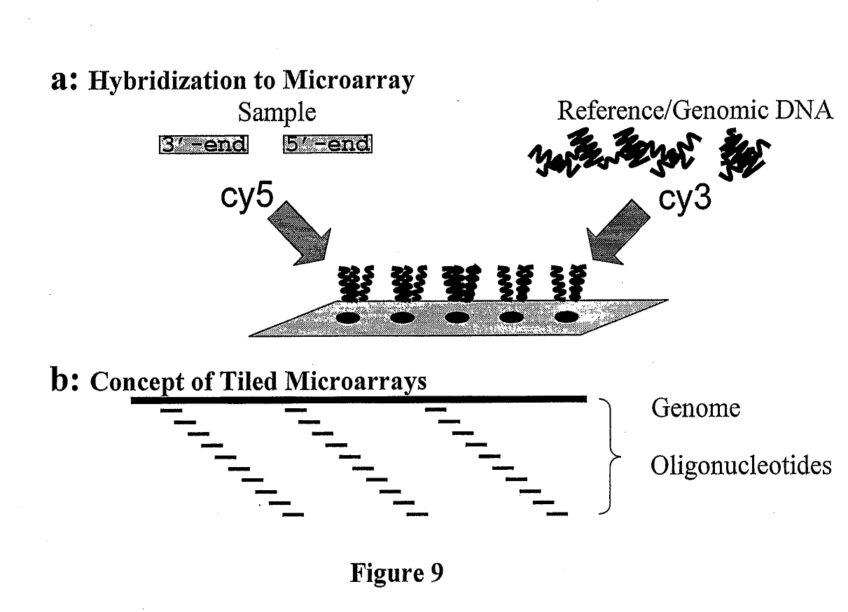

In a different embodiment of the invention, hybridization probes as derived from sequencing tags are used in in situ hybridization experiments, said oligonucleotides. Such experiments include, but are not limited to, the use microarrays FIG. 9). In a preferable embodiment, the microarray is a tiled array, where short oligonucleotides cover partial or entire genomic DNAs, as for example described by Kapranov P. et al., ibid, hereby incorporated herein by reference. Thus the invention provides means for the annotation of sequencing tags by hybridization to microarray, where such a microarray comprises genomic regions. However, the use of hybridization probes derived from sequencing tags is not limited to the use in microarray experiments, as a person trained to the state of the art in the field will know many more applications for the use of hybridization probes including, but not limited to, the ones described by Sambrook J. and Russel D. W. ibid, hereby incorporated herein by reference, or the use of tissue arrays (Sauter G et al., Nature Reviews Drug Discovery 2, 962-972 (2003), hereby incorporated herein by reference).

In just another embodiment, the invention provides means for the preparation of 3′- and 5′-end specific hybridization probes directly from a plurality of RNA molecules. In this embodiment double-stranded linkers having single-stranded overhangs attached to one of the two strands are ligated to the end sequences of the RNA molecules, where one of the strands within the linker will prime the synthesis of the second strand, and where adding terminators into the reaction mixture can control the length of the newly synthesized strand. In the case of preparing probes related to 3′-ends, the probe can be synthesized directly from the RNA template, whereas for the preparation of probes related to the 5′-end, the probes would be prepared from the first-strand cDNA as a template. Many different protocols are known to a person trained to the state of the art to perform the linker ligation step and the following primer extension reaction, including, but not limited to, Shibata Y. et al., Biotechniques 30, 1250-1254 (2001), hereby incorporated herein by reference. In particular, the use of double-stranded linkers having random overhangs or overhangs of defined sequence is of great value to direct the linker to the ends of RNA/DNA molecules. Thus, the invention provides a means for avoiding internal priming. Furthermore, such linkers can be used for the priming of non-polyadenylated RNA, where a linker having an oligo-dT overhang can specifically block the priming from polyadenylated RNA- Such a linker would further have features to block priming of the extension reaction from ployA mRNA, and would have a high affinity label attached to it for selective removal of the ligation product. The invention provides a means for the preparation of end-specific hybridization probes from a plurality of RNAs, which can be used in combination with tiled arrays or in any other hybridization experiment known to a person familiar with the state of the art.

In a different embodiment of the invention, sequence information derived from the concatemers can be used to synthesis specific primers for the cloning of full-length cDNAs. In such an approach, the sequence derived from a given 5′- and 3′-end specific tags allows the design of forward and reverse primers to be used in the amplification reaction. Amplification by the polymerase chain reaction (PCR) can be performed using a template derived from a plurality of RNA obtained from a biological sample and an oligo-dT primer. In the first step the oligo-dT primer and a reverse transcriptase are used to synthesis a cDNA pool. Similarly, the first-strand cDNA synthesis could be primed by the aforementioned ligation of a double-stranded linker having a single-stranded overhang to the 3′-end of RNA In the second step a forward and reverse primers derived from the tags are used to amplify a full-length cDNA from the cDNA pool. Similarly, a specific full-length cDNA can be amplified from an exciting cDNA library. Further, it is within the scope of the invention to use sequence information derived from tags related to genetic elements to design primers for the amplification and cloning of regions within genomic DNA, said promoters or fragments thereof This includes the option to prepare one primer from a GSC-tag and the second tag from a start site of transcription to amplify or clone larger fragments of promoter regions. Many approaches are knowvn to a person familiar with the art for the identification of start sites of transcription including, but not limited to, the CAGE method disclosed in PCT/JP03/07514, and Shiraki T. et al., Prog. Natl. Acad. Sci. USA 100, 15776-15781 (2003), both hereby incorporated herein by reference.

In a different embodiment, the invention relates to a kit, where such a kit would provide the necessary reagents, enzymes and protocols to perform the invention. Thus it can be envisioned that different kits could be provided, where some of the reagents, enzymes or protocols are distinct to adopt the reaction conditions to particular questions or nucleic acid molecules. Such kits could be of value as tools in the filed of life sciences, or forensic assay targeting for the detection and/or identification of certain nucleic acid molecules. Thus it is within the scope of the invention to prepare kits, which would be designed for the detection of specific nucleic acid molecules. In one embodiment, such a selective enrichment would be achieved by the manipulation of single-stranded DNA by the means of subtraction and/or normalization. In a different embodiment, such a selective enrichment would be achieved by the use of specific primers during an amplification step. In a more preferable embodiment, such a selective enrichment would be achieved by the use of specific primers during the rolling-circle amplification step. Furthermore, a kit for the preparation of hybridization probes according to the invention is within the scope of the invention. Similarly, such a kit could provide the necessary means to apply the invention for the purpose of diagnostics.

In conclusion, the invention provides new approaches for the cloning and analysis of sequencing tags by the means of high-throughput sequencing, which will be of great value for the analysis of nucleic acid molecules. The invention provides further the necessary tools to prepare specific hybridization probes as needed for performing in situ hybridization experiments, where related tag sequences would drive the probe design. Thus, the invention is of high importance especially for the annotation of in situ hybridization experiments using tiled arrays, and offers the necessary means for preparing hybridization probes derived from defined regions within nucleic acid molecules.

### EXAMPLES

The present invention will now be further explained in more detail with reference to the following examples. All names and abbreviations as used to describe the invention herein shall have the meaning as known to a person skilled in the art.

### Example 1

### Isolation of RNA

To perform the invention mRNA or total RNA samples can be prepared by standard methods known to a person trained hi the art of molecular biology as for example given in more detail in Sambrook J and Russel D W, ibid, hereby incorporated herein by reference. Furthermore, Carninci P et al. (Biotechniques 33 (2002) 306-309, hereby incorporated herein by reference) described a method to obtain cytoplasmic mRNA fractions. Although the use of cytoplasmic RNA can be preferable, however, the invention is not limited to this method and any other approach for the preparation of mRNA or total RNA should allow for the performance of the invention in a similar manner.

The preparation of mRNA from total RNA or cytoplasmic RNA is preferable but not essential to perform the invention as the use of total RNA can provide satisfying results in combination with the Cap-selection step performed during full-length cDNA library preparation. Here, we have commonly used the Cap-trapper approach, which effectively removes ribosomal RNA from library preparations. Generally speaking, mRNA represents about 1-3% of the total RNA preparations, and it can be subsequently prepared by using commercial kits based on oligo dT-cellulose matrixes. Such commercial kits including, but not limited to, the MACS mRNA isolation kit (ilteny) which provided satisfactory mRNA yields under the recommended conditions when applied for the preparation of mRNA fractions for performing the invention. To perform the invention one cycle of oligo-dT mRNA selection is sufficient as extensive mRNA purification can cause a loss of long mRNAs.

All RNA samples used to perform the invention were analyzed for their ratios of the OD readings at 230, 260 and 280 nm to monitor the RNA purity. Removal of polysaccharides was considered successful when the 230/260 ratio was lower than 0.5 and an effective removal of proteins was obtained when the 260/280 ratio was higher than 1.8 or around 2.0. The RNA samples were further analyzed by electrophoresis in an agarose gel to prove a good ratio between the 28S and 18S rRNA in total RNA preparations (note rRNA size may change for preparation of total RNA from other species than mammalians), and to show the integrity of the RNA fractions.

### Example 2

### cDNA Library Preparation

For the purpose of this example, fill-length cDNA libraries were constructed as described by Carninci P. and Hayashizaki Y., ibid, hereby incorporated herein by reference. This approach makes use of the Cap-trapper approach for full-length cDNA cloning. DNA fragments were cloned into the phage/vector system pFLC, as disclosed in patent application WO 02/070720 A1, hereby incorporated herein by reference.

Phage solutions as prepared to perform the invention were stored in medium containing 7% DMSO and kept at −80° C. However, the invention is not limited to the aforementioned procedure for library preparation, as a person trained to the state of the art knows other methods for the preparation of full-length selected libraries.

### Example 3

### Removal of polyA-tails from cDNA

For the purpose of the invention, cDNAs are prepared from RNA or mRNA fractions as described in Example 2 with the following modifications, which are necessary to remove polyA-tails from cDNA preparations prepared by the use of an oligo-dT primer. Stretches of oligo-dT derived sequences are removed by the means of the Class IIs enzyme GsuI as described by Shibata Y. et al., Biotechniques. 1042 to 1044, 1048-1049 (2001), hereby incorporated herein by reference.

For the first strand synthesis, the following primer is used which has a recognition site for GsuI:

| | |
|---|---|
| Primer GsuI-T14: | |
| 5′-AGAGAGAGAGTCGGAGTTTTTTTTTTTTTTVN | (SEQ ID NO: 1) |

After the first strand cDNA synthesis, the materials are processed as described in Example 2 for the selection of full-length cDNAs by the Cap-Trapper method. In the linker ligation step, the following oligonucleotides were used for linker preparation and to introduce MmeI and XmaJI sites:

5′-Adaptor GS Adaptor C N6-up:

(SEQ ID NO: 2)

5′-GAGAGAGAGACTCGAGACGGCATATCCTAGGTCCGACNNNNNN
5′-Adaptor GS Adaptor C GN5-up:

(SEQ ID NO: 3)

5′-GAGAGAGAGACTCGAGACGGCATATCCTAGGTCCGACGNNNNN
5′-Adaptor GS Adaptor C down:

(SEQ ID NO: 4)

5′-(p)GTCGGACCTAGGATATGCCGTCTCGAGTCTCTCTCTC

Note that the two upper strands are used in a ration of GN5 to GN6 of 4:1.—After preparation of the second strand double-stranded cDNAs were purified as described in Example 2 before being forwarded to GsuI digestion under the following conditions:

| cDNA | X | μl |
|---|---|---|
| 10x buffer B (Fermentas) | 5 | μl |
| 1 u/μl GsuI (Fermentas) | Y | μl (10 u/μg cDNA) |
| 0.1x TE | Z | μl |
| Total volume | 50 | μl* |

*Depending on sample amount, change the reaction volume.

After 1 h incubation at 30° C., the following solutions were added to the reaction:

| | |
|---|---|
| 0.5 M EDTA | 4 μl |
| 10% SDS | 4 μl |
| 20 μg/μl Proteinase K (Qiagen) | 4 μl |

Incubate at 45° C. for 15 min, and continue with Phenol/Chloroform extraction using the following volumes:

| | |
|---|---|
| Phenol/Chloroform | 200 μl |

Centrifagation at room temperature with 15,000 rpm for 3 min, perform back-extraction with 100 μl of 0.1× TE, repeat extraction steps with Chloroform only, and recover the aqueous phase for further purification by microfiltration on a Microcon YM100 (Millipore).

Add 0.1× TE buffer to the cDNA to a final volume of 400 μl, and follow the maker's direction, hereby incorporated herein by reference, for the filtration step. The volume of the recovered sample should be in the range of about 15 μl.

As an option, the 2 bp overhangs created by GsuI can be converted into blunt ends using the 3′ to 5′ exonuclease activity of T4 DNA polymerase. This step is not essential to perform the invention, as also adaptors with a random overhang of 2 bp can be applied in the ligation step. Note, that the blunting step removes 2 bp from the original cDNA

| cDNA | X | μl (>0.1 pmole) |
|---|---|---|
| 0.1x TE | Y | μl |
| Total volume | 14.6 | μl |

Incubate at 65° C. for 5 min, and place on ice immediately. Under the assumption that 100 ng of 2.000 bp cDNA/GsuI are equal to 0.3 pmol end, add the following solutions for the blunting step:

| | | |
|---|---|---|
| 10x T4 DNA Polymerase Buffer (Takara) | 2 | μl |
| 2.5 mM dNTPs (Takara) | 1.4 | μl Vortex |
| 0.1% BSA | 2 | μl Vortex |
| 1 u/μl T4 DNA polymerase | 1 | μl (1 u) |

Mix by pipetting gently up and down, and incubate at 37° C. for 5 min; make sure that the sample is not incubated for a longer time.

Vortex vigorously on ice to inactivate T4 DNA polymerase, and add the following solutions:

| | | |
|---|---|---|
| 0.1x TE | 30 | μl |
| 0.5M EDTA | 1 | μl |
| 10% SDS | 1 | μl |
| Proteinase K (Qiagen) | 2 | μl |
| Total volume | 55 | μl |

Incubate at 45° C. for 15 min, and continue with a Phenol/Chloroform extraction using 50 μl of Phenol/Chloroform, and recover the aqueous phase for fuirther purification by microfiltration on a Microcon YM100 (Millipore). The filtration step follows the maker's instructions, hereby incorporated herein by reference.

To the blunted 3′-end, a double-stranded adaptor has been ligated, where the 3′-adaptor was assembled from the following oligonucleotides:

3′-Adaptor GS 3′ Adaptor C up:

| | |
|---|---|
| 5′-(p)GTCGGACCTAGGAATTGCCGTG | (SEQ ID NO: 5) |

3′-Adaptor GS 3′ Adaptor C Blunt-down:

| | |
|---|---|
| 5′-GATCCACGGCAATTCCTAGGTCCGAC | (SEQ ID NO: 6) |

Note that in a different embodiment of the invention, the cDNA fragments can be amplified by PCR or alike to have larger amounts of DNA for further manipulation. In such a case, primers would be used as selected from the 5′- and 3′-adaptors, and PCR reactions should be performed with a high fidelity DNA polymerase. Although the amplification of the DNA materials is possible after the ligation of the second adaptor, we commonly refrain from amplifying the DNA at this stage as the PCR reaction is highly bias towards shorter DNA fragments, and leads to an uneven distribution of tags within the final library.

For the 3′-adaptor ligation step prepare the following reaction mixture (cDNA: adaptor ratio should be 1:<50):

| cDNA | X | μl |
|---|---|---|
| 0.4 μg/μl GS 3′ Adaptor C | 0.5 | μl (200 ng) |
| 0.1x TE | Y | μl |
| 10 × Ligation Buffer (NEB) | 2 | μl |
| 400 u/μl T4 DNA Ligase (NEB) | 0.5 | μl |
| Total volume | 20 | μl |

Incubate at 16° C. overnight, and inactivate the ligase at 65° C. for 15 min. Optionally, the ligation product can be further purified by Proteinase K treatment, followed by Phenol/Chloroform extraction and ultrafiltration to remove remaining free adaptor. However, those purification steps are not essential to perform the invention, as the ligation product is commonly clean enough for digestion with a standard restriction enzyme, as for the purpose of this example the enzyme XmaJI. Furthermore, free adaptor can be removed after the digestion step.

| | | |
|---|---|---|
| cDNA | 20 | μl |
| 10xbuffer Y+ (Fermentas) | 10 | μl |
| 10xBSA | 10 | μl |
| XmaJI (Fermentas) | X | μl (50 u/μg) |
| 0.1x TE | Y | μl |
| Total volume | 100 | μl |

Incubate at 37° C. for 1 h, and inactivate the enzyme by heating to 65° C. for 15. min. Further purify the cDNA fragments by Proteinase K treatment, Phenol/Chloroform extraction, followed by PEG precipitation. The PEG precipitation is applied here to remove the very short fragments cut off from the adaptors and free adaptors. For the purpose of this example, short fragments were removed by PEG precipitation, as the adaptors used here were not labeled by a selective binding substance e.g. biotin or digoxigenin. Example 10 describes the use of labeled linkers in fragment purification. For the precipitation by PEG prepare the following:

| | | |
|---|---|---|
| cDNA | 150 | μl |
| 0.1x TE | 50 | μl |
| 20% PEG8000 | 250 | μl |
| 0.1M MgCl₂ | 50 | μl |
| Total volume | 500 | μl |

Leave at room temperature for 10 min before centrifugation with 15,000 rpm at room temperature for 10 min, remove the supernatant completely, and rinse the tube wall well with 20 μl of TE to make sure that the entire pellet is re-suspended. Leave the tube for a while at room temperature before transfering the solution into a new siliconized tube. Wash the original tube again with 20 μl of TE to make sure that the sample is recovered completely. Combine the cDNA solutions in one tube (about 40 μl in total). Optionally, remaining 3′ adaptors can be further removed by gel filtration on a CL4B column (Amersham Biosciences).

### Example 4

### Preparation of GSC-Tags

For the preparation of GSC-Tags aforementioned cDNA fragments are circulated by self-ligation using the cohesive ends created by digestion with XmaJI. It is important to perform this ligation step in a large volume (1 ng DNA/μl) to favor self-ligation over inter-molecular ligation. For the reaction setup the following solutions (split the cDNA over various tubes where necessary to achieve a high dilution):

| cDNA | X | μl (1 μg) |
|---|---|---|
| 10x Ligation Buffer (NEB) | 100 | μl |
| 400 u/μl T4 DNA Ligase (NEB) | 50 | μl (20,000 units, 20 u/ng) |
| H₂O | Y | μl |
| Total volume | 1000 | μl |

Incubate at 23° C. for 2 h in a water bath, before inactivating the ligase at 65° C. for 10 min.

The ligation product was firther purified using a “QLAquick PCR Purification Kit” (Qiagen) according to the maker's directions, hereby incorporated herein by reference.

Remaining unligated DNA, and thus linear DNA, in the ligation mixture was removed by Exonuclease III treatment. Exonuclease III acts only on double-stranded linear DNA and does not cut the circular DNA under the controlled condition. For Exonuclease III digestion set up the following reaction:

| Self-ligation products | X | μl (1.5 μg) |
|---|---|---|
| 10x Exonuclease III buffer (Epicentre) | 30 | μl |
| 200 u/μl Exonuclease III (Epicentre) | 3 | μl (400 u/μg) |
| H₂O | Y | μl |
| Total volume | 300 | μl (5 ng/μl) |

Incubate at 37° C. for 30 min and add:

| | |
|---|---|
| 0.5M EDTA | 6 μl |

Inactivate Exonuclease II at 65° C. for 15 min, cool on ice, and purify DNA by Proteinase K digestion, Phenol/Chloroform extraction, and ethanol precipitation as described above. Dissolve the remaining pellet in 15 μl of 0.1× TE.

At this stage usually only very small amounts of DNA are available for the further processing, and an amplification step in essential in most cases to have sufficient DNA amounts for tag cloning. This is in particular true, where the cDNA was not amplified by PCR after the second linker ligation step (see above). As it is desirable here to amplify only circular DNA, this amplification step makes use of the so-called rolling-circle amplification including but not limited the TempliPhi Amplification Kit from Amersham Biosciences (Product No. 25-6400-10, the instructions of which are hereby incorporated herein by reference). This kit makes use of the Phi29 DNA polymerase and random priming by hexamers to perform the amplification reaction. Commonly as little as 1 ng of circular DNA is sufficient for amplification, where the reactions can yield up to 1 μg of DNA after 4 to 12 h. As the reaction is sensitive to the use of too much template in the reaction, it can be preferable to run multiple reactions in parallel. Otherwise, amplification reactions are performed according to the maker's directions. Note that the reaction product can be very viscous as it contains very long stretches of DNA.

Amplification products are directly forwarded to digestion with the Class IIs enzyme, for the purpose of this example MmeI. Where needed, viscous DNA solutions can be diluted to allow for a better pipetting. For the digestion with MmeI set up the following reaction:

| Amplified DNA | X | μl (20 μg) |
|---|---|---|
| 3.2 mM SAM* | 20 | μl (64 μM) |
| 10xNEB buffer 4 (NEB) | 100 | μl 2 u/μl |
| MmeI (NEB) | 15 | μl (1.5 u/μg, 30 u) |
| H₂O (Invitrogen) | Y | μl |
| Total volume | 1000 | μl |

*S-Adenosylmethionine (NEB)

Incubate at 37° C. for 1 h, and purify reaction fixture by Proteinase K digestion, Phenol/Chloroform extraction, and precipitation under the following conditions:

Add to about 600 μl DNA solution:

| | | |
|---|---|---|
| 1 μg/μl Glycogen | 3 | μl |
| 5 M NaCl | 30 | μl |
| Isopropanol | 600 | μl |

Incubate at −20° C. for more than 30 min, and centrifugate at 15,000 rpm at 4° C. for 15 min before washing the pellet twice with 80% ethanol, and dissolve the precipitant in 50 μl H₂O. As MmeI digestion can be insufficient, analyze the reaction product by gel electrophoresis before continuing the process.

The short GSC-tags as cut out with MmeI have to be separated from the remaining cDNA fragments. In theory, a GSC-tag has some 58 bp (2 times 20 bp cut off from cDNA ends plus 18 bp from the three recognition sites derived from the linkers), where the length of the tag may vary within a range of some 4 to 8 bp as MmeI digestion in not always precise. However, with some 58 bp in length the GSC-tags are much shorter than cDNA fragments but still longer than the adaptors used in the earlier preparation steps. Thus the GSC-tags can by purified by size-selection.

GSC-tags were separated from other cDNAs by agarose gel electrophoresis. For the electrophoresis proceed as following:

### Sample Preparation:

| | | |
|---|---|---|
| Sample DNA | 20 | μl (~800 ng) |
| 10% SDS | 1.5 | μl (final ~0.5%) |
| 0.1x TE | 3.5 | μl |
| 6x Dye (TAE) | 5 | μl |
| Total volume | 30 | μl |

Gel: 5% SeaPlaque/1×TAE/ EtBr+, Mupid Mini Gel

Buffer: 1× TAE bufferEtBr+

Run: Mupid System, 50 V, 150 min

After electrophoresis, cut out GSC-tags as compared to an appropriate size marker using a UV transilluminator at 365 nm. When cutting out the gel slices, make sure to keep their size as small as possible. Furthermore, it is important to cut precisely the band around 58 bp, where it is preferable to cut sharp around the band rather than retrieving as much DNA as possible.

Transfer gel pieces into a tube, add 300 μl TE buffer, and keep the tube on ice for 1 h or overnight to elute the GSC-tags. GSC-tags were further retrieved from the gel pieces by filtration on a Micro Spin Column (Amersham) according to the maker's directions, hereby incorporated herein by reference. The GSC-tags should be eluted in a volume of about 700 μl.

After the gel purification step, GSC-tags are further concentrated on Microcon YM-10 membrane (Millipore) according to the maker's directions, hereby incorporated herein by reference. About 20 μl of eluted DNA should be recovered after this step.

### Example 5

### Concatenation of GSC-Tags

Individual GSC-tags are ligated into concatemers using their N2 cohesive ends out of the MmeI digestion step. Although 16 different overhangs can occur, the complexity of most samples is sufficient to allow for the concatenation of the different GSC-tags. However, in some cases, it can be advisable to blunt the GSC-tags before the concatenation step, although this leads to a shortening of the tags. An example for the blunting of MmeI sites is given below.

For the ligation reaction mix the following components in a 0.2 μl PCR tube:

| GSC-tag fragments | X | μl (300-500 ng) |
|---|---|---|
| 10x buffer (Takara) | 1 | μl |
| T4 DNA Ligase (Takara) | 1 | μl |
| 0.1x TE | Y | μl |
| Total volume | 10 | μl |

Incubate ligation reaction at 16° C. for 5 min. Note that the ligation reaction should not exceed 5 min. Add 0.5 μl of 10% SDS before inactivating the ligase at 65° C. for 3 min.

To assure for a satisfying number of GSC-tags within each concatemer, it is advisable—although not essential—to perform a size fractionation of the concatenation products, where we commonly isolate fragments of more than 500 bp.

Size fractionation of concatemers is commonly performed by agarose gel electrophoresis under the following conditions:

Gel: 0.8% SeaPlaque/1×TAE/EtBr+

Buffer: 1× TAE buffer/EtBr+

Run: “50V, 170 min, at 4° C.

Cut out fragments of about 500 to 700 bp, and elute the DNA as described above. The DNA can be further concentrated using a Micro Spin Column (micron YM-10, Amersham Biosciences).

For the purpose of this example, the concatenation products were blunted for ligation into the vector. Although vectors with N2 overhangs can be prepared, it is preferable to clone blunted concatemers to assure cloning of all possible combinations. For the blunting reaction, setup the following:

| Concatemers | X | μl |
|---|---|---|
| H₂O (Invitrogen) | Y | μl |
| 10x buffer (Takara) | 18 | μl |
| 0.1% BSA (Takara) | 18 | μl |
| 1.7 mM dNTPs (dilute Takara 2.5 mM) | 18 | μl |
| Total volume | 162 | μl |

Incubate at 65° C. for 5 min before placing on ice for 1 min, then add:

| | |
|---|---|
| 4 u/μl T4 DNA Polymerase (Takara) | 18 μl (72 u, 4 u/μg DNA) |
| Total volume | 180 μl (18 μg/180 μl = 100 ng/μl) |

Incubate at 37° C. for 5 min in a water bath without water circulation. After the incubation inactivate T4 DNA polymerase by vigorous vortexing for about 10 min. From there proceed by digestion with Proteinase K, extraction with Phenol/Chloroform, and Chloroform.

### Example 6

### Preparation of Vector pGSC for Ligation Step

For the purpose of this example the vector pGSC is used to perform the invention, however the invention can be performed using many other vector as well. As for the use of blunt end ligation of GSC-tags, the vector is digested with the restriction enzyme Hpa I. For the digestion the following reaction is setup:

| pGSC plasmid DNA | X | μl (20 μg) |
|---|---|---|
| 10x NEBuffer 4 (NEB) | 50 | μl |
| HpaI (NEB) | 30 | μl (5000 u/ml) |
| H₂O | Y | μl |
| Total volume | 500 | μl (40 ng/μl) |

Incubate at 37° C. for 2 h, and check an aliquot by gel electrophoresis to assure complete digestion. In case that the digestion was complete, purify the linear DNA by Proteinase K digestion, Phenol/Chloroform extraction, Chloroform extraction and ethanol precipitation. The DNA should finally be dissolved in 40 μl H₂O.

To avoid self-ligation of the vector a de-phosphorylation by calf intestine alkaline phosphatase can be advisable. To perform the reaction setup the following:

| | | |
|---|---|---|
| pGSC/HpaI | 40 | μl (20 μg, 35.2 pmole) |
| 10x Buffer (Takara) | 10 | μl |
| CIP (Takara) | X | μl (140 u, 4 u/pmole) |
| H₂O | Y | μl |
| Total volume | 100 | μl |

Incubate at 37° C. for 15 min before inactivating the enzyme at 50° C. for 15 min. Purify the DNA by Proteinase K digestion, Phenol/Chloroform extraction, and ethanol precipitation. Finally dissolve DNA pellet in 80 μl H₂O.

Furthermore, it can be advisable to purify the DNA in an agarose gel under the following conditions:

### Sample Preparation:

| | | |
|---|---|---|
| pGSC/HpaI/CIP | 80 | μl |
| 6x Dye (TAE) | 20 | μl |
| Total volume | 100 | μl |

Gel: 0.8% SeaPlaque/1× TAE/EtBr+, Mupid small gel using wide wells

Buffer: 1× TAE buffer/EtBr+

Run: 35V, 160 min

After the electrophoresis, cut out the band corresponding to 2,800 bp as compared to an appropriate size marker using a transilluminator (365 nm). The DNA can be eluted from the gel pieces by the following steps:

Melt gel slices at 65° C. for 5 min, and confirm that all gel pieces melted completely. Add to some 800 μl solution β-agarasebuffer mix (NEB), and incubate at 42° C. for 5 h. Add 5M NaCl at 1/9 of the reaction volume, and extract with Phenol/Chloroform. Precipitate the DNA out of the aqueous phase waith isoporpanol, wash twice with 80% ethanol, and dissolve the pellet in 30 μl H₂O. About 5 μg of linerized vector may be gained, which can be stored at −20° C.

### Example 7

### Ligation of GSC-Tag-Concatemers into Vector pGSC

Purified concatemers as prepared according to Example 5 are ligated into vector pGSC/HpaI/CIP prepared according to Example 6. For the ligation reaction setup the following precipitation to concentrate the DNA:

| | | |
|---|---|---|
| Concatenated GSC-tags | X | μl (~200 ng) |
| pGSC/HpaI/CIP vector | Y | μl (260 ng) |
| 5M NaCl | Z | μl (final concentration 250 μM) |
| Isopropanol | A | μl |

Ligation ratio: pGSC vector:Concatenated GSC-tag=1:2 (mol)

Incubate at −20° C. for more than 30 min before collecting the precipitate by centrifugation at 15.000 rpm for 15 min at 4° C. Discard the supernatant and wash the pellet twice with 80% ethanol before dissolving the pellet with 26 μl 0.1× TE buffer. For the ligation reaction setup:

| | | |
|---|---|---|
| Concatemers/pGSC vector | 5 | μl |
| 2 × Ligation Mix (Nippon Gene) | 5 | μl |
| Total volume | 10 | μl |

Incubate at 16° C. for 30 min before inactivation of the ligase, and then inactive the ligase at 65° C. for 10 min. Commonly, the ligation product is directly used for transformation of bacteria, although it can be advantageous to purify the ligation product for longer storage or to de-salt the reaction mixture for electroporation.

For transformation we commonly use the following setup, although other approaches or bacteria can be used as well at this stage:

| | |
|---|---|
| Sample: | 5 ng/μl, 2 μl |
| Bacterial: | DH10B T1 phase resistance (Invitrogen), 20 μl |

Commonly we prefer to use electroporation for the transformation step using Cell-Porator (Invitrogen) according to the transformation procedures described in the manufacturer's manual, hereby incorporated herein by reference. After electroporation spread some 10 μl of the bacteria on LB medium containing chloramphenicol (12.5 μl/μl). Individual colonies can be obtained after overnight grow at 37° C. Remaining bacteria not plated onto the selective media can be stored as glycerol stocks at −80° C.

### Example 8

### Insert Size Check for GSC-Tag Libraries

It can be of value to check the average insert size of the GSC-tag libraries before initiating high-throughput sequencing. The insert size of GSC-libraries can be determined by the following reaction setup.

| Plasmid | X | μl (200 ng) |
|---|---|---|
| 10x NEB Buffer 2 (NEB) | 2 | μl |
| 100x BSA (NEB) | 0.2 | μl |
| 20 u/μl XbaI | 0.2 | μl (4 u) |
| H₂O | Y | μl |
| Total volume | 20 | μl (10 ng/μl) |

Incubate at 37° C. for 2 h, and take an aliquot agarose gel electrophoresis:

| | | |
|---|---|---|
| Sample DNA | 5 | μl |
| 0.1x TE | 5 | μl |
| 6x Dye (for TBE) | 2 | μl |
| Total volume | 12 | μl |

Gel: 1% Agarose (EtBr+, 1× TBE), Mupid gel

Buffer: 1×TBE buffer

Electrophoresis system: Mupid

Run: 100 V, 30 min

### Example 9

### Purification of Oligonucleotides for Library Preparation

Oligonucleotides as used in these Examples have been obtained from Invitrogen, and were before use purified by 10% polyacrylamide/7M Urea/1×TBE gel electrophoresis.

### Example 10

### Capture of PCR Products by Streptavidine Coated Magnetic Beads

In cases where biotinylated linkers or PCR primers have been used, reaction products can be attached to magnetic beads via a Streptavidin/biotin interaction. Commonly, we use here Takara MAGNOTEX-SA (Takara) according to the maker's directions, hereby incorporated herein by reference. For sample preparation mix the following:

| | | |
|---|---|---|
| Purified PCR product | 100 | μl (~5 μg) |
| 2x Binding Buffer (Takara) | 100 | μl |
| Total | 200 | μl |

Magnetic beads should be prepared from the slurry, from which

| | |
|---|---|
| MAGNOTEX-SA | 150 μl |

are placed on a Magnetic stand for 2 min. remove supernatant, then add:

| | |
|---|---|
| 1x Binding Buffer | 200 μl |

vortex gently, apply magnetic force, remove supernatant, and repeat washing step with 2×Binding Buffer (Takara), replace 2×Binding Buffer by 1×Binding Buffer.

Add some 200 μl of PCR product to the magnetic beads, and incubate for 15 min at room temperature under ongoing agitation. Apply the magnetic force and remove the supernatant, and wash the magnetic beads three times with 250 μl of 1× Binding Buffer.

cDNA fragments are released from the beads by digestion with an appropriate restriction endonuclease. For the purpose of this example, the enzyme XmaJI was used under the same conditions as described in Example 3.

### Example 11

### Determination of End-Sequences

After the titer check, bacterial clones were collected by commercially available picking machines (Q-bot and Q-pix; Genetics) and transferred to 384-microwell plates. Transformed E. coli clones holding vector DNA were divided from 384-microwell plates and grown in four 96-well plates. After overnight growth, plasmids were extracted either manually (Itoh M. et al., Nucleic Acids Res. 25 (1997) 1315-1316, hereby incorporated herein by reference) or automatically (Itoh M. et al., Genome Res. 9 (1999) 463-470, hereby incorporated herein by reference). Sequences were typically run on a RISA sequencing unit (Shimadzu) or a Perkin Elmer-Applied Biosystems ABI 3700 in accordance with standard sequencing methodologies such as described by Shibata K. et al., Genome Res. 10 (2000) 1757-1571, hereby incorporated herein by reference. Sequencing was alternatively performed using primers nested in the flanking regions of the cloning vector and a BigDye Terminator Cycle Sequencing Ready Reaction Kit v1.1 (Applied Biosystems, Cat. No. 4337449) and an ABI3700 (Applied Biosystems) sequencer according to the manufacture's product descriptions, hereby incorporated herein by reference.

Standard primers as used for vectors of the pFLC or pGSC family included:

| | |
|---|---|
| M13 Reverse primer: | |
| 5′-CAGGAAACAGCTATGAC | (SEQ ID NO: 7) |
| M13 (−20) Forward primer: | |
| 5′-GTAAAACGACGGCCAG | (SEQ ID NO: 8) |

### Example 12

### Characterization of Sequence Tags

Individual sequence tags can be analyzed for their identity by standard software solutions to perform sequence alignments like NCBI BLAST (http://www.ncb.nlm.nih.gov/BLAST/), FASTA, available in the Genetics Computer Group (GCG) package from Accelrys Inc. (http://wwv.accelrys.com/) or alike. Such software solutions allow for an alignment of specific sequence tags among one another to identify unique or non-redundant tags, which can be further used in database searches.

### Example 13

### Mapping of Sequencing Tags to the Genome

Specific sequence tags obtained as describe in this Example can be used to identify transcribed regions within genomes for which partial or entire sequences were obtained. Such a search can be performed using standard software solutions like NCBI BLAST (http://www.ncbi.nlm.nih.gov/BLAST/) to align specific sequence tags to genomic sequences. In the case of large genomes like those from human, rat or mouse it may be necessary to extend the initial sequence information obtained from concatemers. The use of extended sequences allows for a more precise identification of actively transcribed regions in the genome.

### Example 14

### Statistical Analysis of Sequence Tags

Sequence tags obtained from the same plurality of mRNAs in a sample or nucleic acid fragments within the same cDNA library can be analyzed by a standard software solution like NCBI BLAST (http://www.ncbi.nlm.nih.gov.BLAST/) to identify non-redundant sequence tags. All such non-redundant sequence tags can then be individually counted and further analyzed for the contribution of each non-redundant tag to the total number of all tags obtained from the same sample. The contribution of an individual tag to the total number of all tags should allow for a quantification of the transcripts in a plurality of mRNAs in the sample or a cDNA library. The results obtained in such a way on individual samples can be further compared with similar data obtained from other samples to compare their expression patterns.

### Example 15

### Identification of Transcriptional Start Sites

5′ end specific sequence tags, which could be mapped to genomic sequences, allow for the identification of regulatory sequences. In a gene the DNA upstream of the 5′ end of transcripted regions usually encompasses most of the regulatory elements, which are used in the control of gene expression. These regulatory sequences can be further analyzed for their functionality by searches in databases, which hold information on binding sites for transcription factors. Publicly available databases on transcription factor binding sites and for promoter analysis include:

Transcription Regulatory Region Database (TRRD)

(http://www.mgs.bionet.nsc.ru/mgs/dbases/trrd4/)

TRANSFAC (http://transfac.gbf.de/TRANSFAC/)

TFSEARCH (http:www.cbrc.jp/research/db/TFSEARCH.html)

PromoterInspector provide by Genomatix Software (http://www.genomatix.de/)

1. A method for preparing DNA fragments comprising sequences corresponding to two opposite end regions of a linear nucleic acid molecule, comprising the steps of:
(a) creating a linear DNA molecule from a nucleic acid molecule;
(b) ligating linkers to two opposite ends of the linear DNA molecule, wherein such the linkers contain a cloning site and a recognition site for a restriction endonuclease that cleaves at a site outside its recognition site and within the linear DNA molecule;
(c) circularizing the linear DNA molecule by closing the linear DNA molecule at the cloning site with the linkers so as to form a circular DNA molecule;
(d) digesting the circular DNA molecule with the restriction endonuclease so as to cut out a DNA fragment from the circular DNA molecule, wherein the DNA fragment comprises opposite end regions of the linear DNA molecule; and
(e) isolating the DNA fragment.

2-47. (canceled)

48. The method according to claim 1, wherein the nucleic acid molecule is selected from the group consisting of a DNA, cDNA, genomic DNA, RNA, mRNA having poly(A) tail, mRNA lacking poly(A) tail and any mixture thereof.

49. The method according to claim 48, wherein the nucleic acid molecule of step (a) is mRNA having poly(A) tail, and wherein step (a) comprises converting the mRNA into a complementary DNA by the means of a reverse transcriptase and a primer, wherein the primer contains a Class IIS or Class III recognition site for removing stretches of oligo-dT used in the priming of the reverse transcription reaction from the. RNA which is an RNA having poly(A)tail.

50. A method for preparing DNA fragments comprising sequences corresponding to two opposite end regions of an RNA, comprising the steps of:
(a) creating a linear DNA molecule from an RNA;
(b) ligating linkers to two opposite ends of the linear DNA molecule, wherein the linkers contain a cloning site and a recognition site for a restriction endonuclease that cleaves at a site outside its recognition site and within the linear DNA molecule;
(c) circularizing the linear DNA molecule by closing the linear DNA molecule at the cloning site with the linkers so as to form a circular DNA molecule;
(d) digesting the circular DNA molecule with the restriction endonuclease so as to cut out a DNA fragment from the circular DNA molecule, wherein the DNA fragment comprises opposite end regions of the linear DNA molecule; and
(e) isolating the DNA fragment, wherein step a) above comprises:
(i) preparing a double-stranded linker having a single-stranded overhanging region, wherein the single-stranded overhanging region is complementary to the 3′-end sequence of the RNA;
(ii) hybridizing the single-stranded overhanging region to the 3′-end sequence of the RNA so as to ligate the double-stranded linker to the 3′-end of the RNA,
(iii) extending a strand complement to the RNA from the 3′ end of the overhang region of the linker with a reverse transcriptase and
(iv) separating a linear DNA molecule from the reverse transcription product.

51. The method according to claim 50, wherein the RNA is enriched by the Cap Trapper method or Oligo capping method, and thereby a full length cDNA is prepared in step a).

52. The method according to claim 49, wherein any complementary sequences derived from a poly(A) tail of the mRNA are removed from the linear cDNA molecule.

53. The method according to claim 1, wherein the restriction endonuclease is selected from the group consisting of the Class US, Class IIG, Class III restriction enzymes, Gsu I, MmeI, Bpm I, Bsg I, EcoP15I, and any mixture thereof.

54. The method according to claim 1, wherein the linkers are attached to a selective binding substance to allow for enrichment by such binding.

55. The method according to claim 54, wherein the selective binding substance is selected from the group consisting of biotin and digoxigenin, and a high affinity binding substance bound to the selective binding substance is selected from the group consisting of avidin, streptavidin, a derivative of avidin or streptavidin, and an anti-digoxigenin antibody.

56. The method according to claim 1, where at least one of the linkers contains sequence elements used for labelling the DNA fragment.

57. The method according to claim 1, wherein the linear DNA fragments are removed from the circular DNA molecule by the means of an exonuclease.

58. The method according to claim 57, wherein the exonuclease is exonuclease III, exonuclease I, or any mixture thereof.

59. The method according to claim 1, further comprising the step of amplifying the circular DNA molecule.

60. The method according to claim 59, wherein the step of amplifying the circular DNA molecule is a rolling circle reaction.

61. A method for preparing a concatemer, comprising ligating the DNA fragments to each other, wherein the DNA fragments are prepared by the method of claim 1.

62. Vector pGSC.

63. A method for obtaining information on the end sequences of a linear nucleic acid molecule, comprising some or all steps of:
preparing the DNA fragments by the method according to claim 1,
preparing a concatemer by ligating the DNA fragments to each other, and
sequencing the concatemer so as to obtain information on the end sequences of the linear nucleic acid molecule.

64. The method according to claim 1, wherein the DNA fragment is derived from a mixed sample.

65. The method according to claim 64, wherein the origin of the DNA fragment in the mixed sample can be tracked by a label which is a short specific sequence in a spacer which is derived from the linker sequences.

66. A method for priming a reverse transcription reaction, comprising the steps of:
(a) preparing a double-stranded linker having a single-stranded overhanging region, wherein the single-stranded overhanging region is complementary to a 3′ -end sequence of an RNA;
(b) hybridizing the single-stranded overhanging region to the 3′-end sequence of an RNA so as to ligate the double-stranded linker to the 3′-end of the RNA; and
(c) extending a strand complement to the RNA from the 3′ end of the overhang region of the linker with a reverse transcriptase.

67. The method according to claim 66, wherein the overhanging part of the linker is comprised of oligo-dT.

68. The method according to claim 66, wherein the overhang part of the linker has random sequence.

69. A method for separating an mRNA having poly(A) tail and an mRNA having no poly(A) tail, comprising the steps of:
(a) preparing double-stranded linkers having a single-stranded overhanging region, wherein the overhang region of the first linker has oligo-dT and wherein the 3′-end of the oligo-dT overhang region is blocked and wherein the overhang region of the second linker has a random sequence and the 3′-end of the random sequence is not blocked;
(b) hybridizing the single-stranded overhanging regions to the 3′-end sequence of an RNA so as to ligate the double-stranded linker to the 3′-end of the RNA in one or more ligation reactions;
(c) perform the reverse transcription reaction so that a strand is extended from the 3′ overhang region of the second linker;
(d) selecting the RNA ligated to the first double-stranded linker; and
(e) separating a linear DNA molecule from the reverse transcription product derived from the second linker.

70. The method according to claim 66, wherein the linker is attached to a selective binding substance used for the fractionation of RNAs.

71. The method according to claim 66, further comprising the step of attaching the linker to a high affinity selective binding substance so as to allow for enrichment.

72. The method according to claim 71, where the selective binding substance is selected from the group consisting of biotin and digoxigenin, and a high affinity selective binding substance bound to the selective binding substance is selected from the group consisting of avidin, streptavidin, a derivative of avidin or streptavidin, or an anti-digoxigenin antibody.


SEQUENCE LISTING (14 sequences)

SEQ ID NO: 1 - DNA, 32 bp, Artificial
  Primer GsuI-T14
agagagagagtcggagttttttttttttttvn

SEQ ID NO: 2 - DNA, 43 bp, Artificial
  5′-Adaptor GS Adaptor C N6-up
gagagagagactcgagacggcatatcctaggtccgacnnnnnn

SEQ ID NO: 3 - DNA, 43 bp, Artificial
  5′-Adaptor GS Adaptor C GN5-up
gagagagagactcgagacggcatatcctaggtccgacgnnnnn

SEQ ID NO: 4 - DNA, 37 bp, Artificial
  5′-Adaptor GS Adaptor C down
gtcggacctaggatatgccgtctcgagtctctctctc

SEQ ID NO: 5 - DNA, 26 bp, Artificial
  3′-Adaptor GS 3′ Adaptor C up
gtcggacctaggaattgccgtggatc

SEQ ID NO: 6 - DNA, 26 bp, Artificial
  3′-Adaptor GS 3′ Adaptor C Blunt-down
gatccacggcaattcctaggtccgac

SEQ ID NO: 7 - DNA, 17 bp, Artificial
  M13 Reverse primer
caggaaacagctatgac

SEQ ID NO: 8 - DNA, 16 bp, Artificial
  M13 (-20) Forward primer
gtaaaacgacggccag

SEQ ID NO: 9 - DNA, 111 bp, Artificial
  pGSC
tgtaaaacgacggccagtgaattgcgcgcaattaaccctcactaaagggaacaaagatgtgtaactataacggtcctaaggtagcgagtcgactctagagttaacttcgaa

SEQ ID NO: 10 - DNA, 133 bp, Artificial
  pGSC
aagcttctcttcgaagttaactctagaggatcctgccatttcattacctctttctccgcacccgacatagatgcatcgcccctatagtgagtcgtattacatagctgtttcctgtgtgaaattgttatccgct

SEQ ID NO: 11 - DNA, 16 bp, Artificial
  double stranded linker having a random single stranded overhang
gagagagagactcgag

SEQ ID NO: 12 - DNA, 16 bp, Artificial
  double stranded linker having a random single stranded overhang
ctctctctctgagctc

SEQ ID NO: 13 - DNA, 16 bp, Artificial
  double stranded linker having a single stranded oligo dT overhang
gagagagagactcgag

SEQ ID NO: 14 - DNA, 16 bp, Artificial
  double stranded linker having a single stranded oligo dT overhang
ctctctctctgagctc